(19) United States
(12) Patent Application Publication
(10) Pub. No.: US 2010/0233225 A1
(43) Pub. Date: Sep. 16, 2010

(54) Method of surface coating devices to incorporate bioactive molecules

(76) Inventor: Bingyun Li, Morgantown, WV (US)

Correspondence Address: WEST VIRGINIA UNIVERSITY RESEARCH CORPORATION, 886 CHESTNUT RIDGE ROAD, P.O. BOX 6224, MORGANTOWN, WV, 26506-6224, US

(21) Appl. No.: 12/069,526

(22) Filed: Feb. 11, 2008

Related U.S. Application Data
(60) Provisional application No. 60/900,498, filed on Feb. 9, 2007.

(51) Int. Cl. A61K 9/00; A61K 38/20; A61K 38/19; A61K 38/00; A61K 31/7088; A61K 35/00; A61P 43/00; A61K 31/7048

(52) U.S. Cl. 424/422; 424/85.2; 424/85.1; 512/2; 514/44 R; 424/93.1; 514/40

(57) ABSTRACT

A first embodiment is a coating for a surface comprising an electrostatic self-assembly creating one or more coating layer(s); (i) wherein one or more high molecular weight bioactive molecule(s) is bound to a protein carrier wherein said protein carrier or said bound bioactive molecule is further bound to one or more of said coating layer(s) and/or (ii) wherein one or more low molecular weight bioactive molecule(s) is/are incorporated within one or more of said layers or later introduced to said layer or layers by post-treatment. A second embodiment is a method of coating a surface comprising introducing said surface into a solution of one or more positively charged water-soluble polyelectrolytes at a pH ranging from about 2 to about 12 and at a temperature ranging from about 4° C. to about 80° C. and incubating said surface for about 0.5 to about 60 minutes; Rinsing said surface with said buffer solution or water to remove weakly bound material on said surface and dry by means known to one skilled in the art; Introducing said surface into a solution of one or more negatively charged water-soluble polyelectrolytes at a pH ranging from about 2 to about 12 and at a temperature ranging from about 4° C. to about 80° C. and incubating said surface for about 0.5 to about 60 minutes; and Rinsing said surface with said buffer solution or water to remove weakly bound material on said surface and passively or actively drying said surface.

### CROSS-REFERENCE TO RELATED APPLICATIONS

This application claims priority from U.S. provisional application 60/900,498.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

### BRIEF DESCRIPTION OF FIGURES

The following Figures are for illustrative purposes only and are not to scale:

FIG. 1 is an illustration of charged assembly on an implant device with drug incorporation.

FIG. 2 is a photograph of bioactive coating on stainless steel implants.

FIG. 3 is an illustration of the strategy of multifunctional nanocoating.

FIG. 4 is a graph of number of bilayers as a function of drug loaded.

FIG. 5 is a bar chart of pH of drug solution as a function of drug loaded.

FIG. 6 are graphs of MB and drug loaded as a function of time.

FIG. 7 is a graph of cefazolin loaded at various pHs as a function of time.

FIG. 8 is a graph of drug release as a function of time.

FIG. 9 is a bar graph of drug release as a function of pH of the medium of release.

FIG. 10 are graphs of thickness and absorbance as a function of number of bilayers. The scale bar in the inset AFM images is 200 nm.

FIG. 11 are pictures of synthetic polypeptide fibrillar structures formed on quartz slides.

FIG. 12 are pictures of synthetic polypeptide fibrillar structures formed on stainless steel implants.

FIG. 13 is absorbance as a function of wavelength to monitor multilayer coating with RGD. The inset is the fluorescence image of the coating.

FIG. 14 is RGD release as a function of time for multilayer coatings. The inset is the picture of the coated quartz slide before (left) and after (right) drug release.

FIG. 15 is a graph of osteoblast cell attachment shown as cell numbers attached as a function of time. The insets are the fluorescence images of osteoblast cells on the coatings after 4 h culture.

FIG. 16 is a graph of osteoblast cell attachment shown as cell numbers attached as a function of time.

FIG. 17 is a graph of antibacterial property shown as zone inhibition as a function of the number of bilayers.

FIG. 18 is a chart of antibacterial property with absorbance as a function of time.

### DETAILED DESCRIPTION OF THE INVENTION

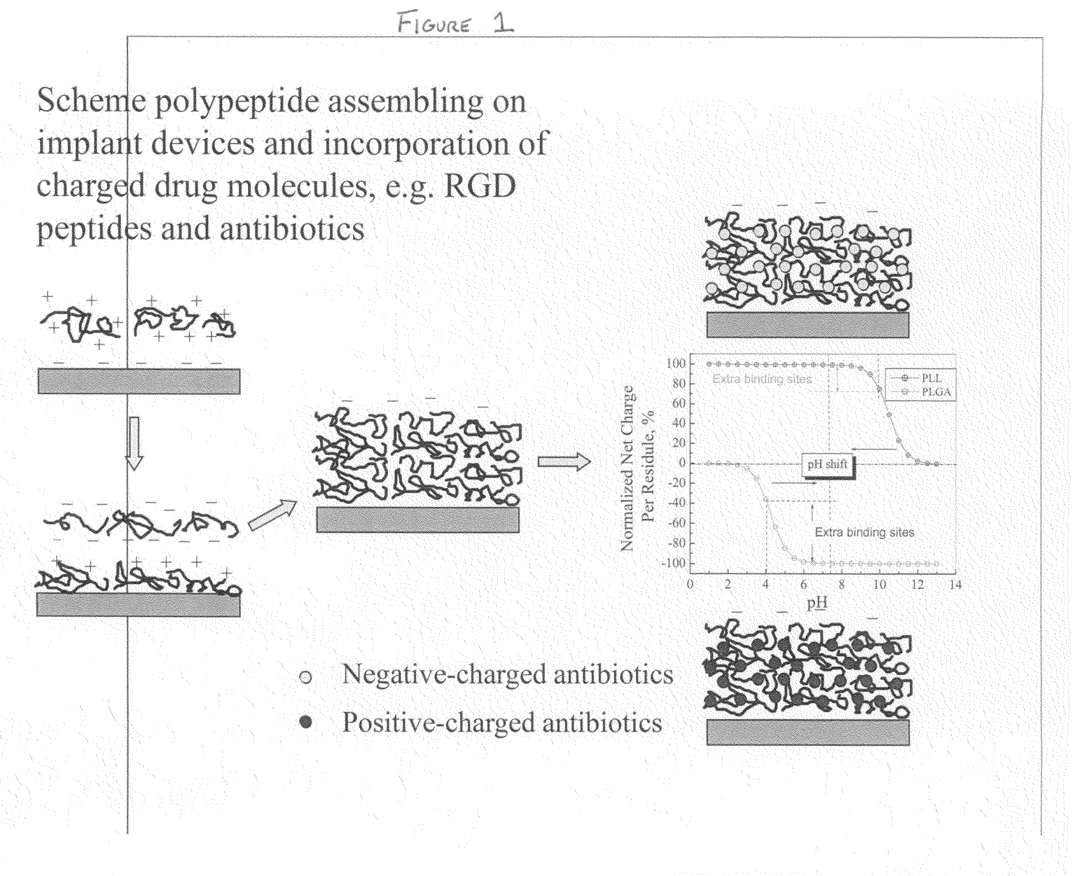

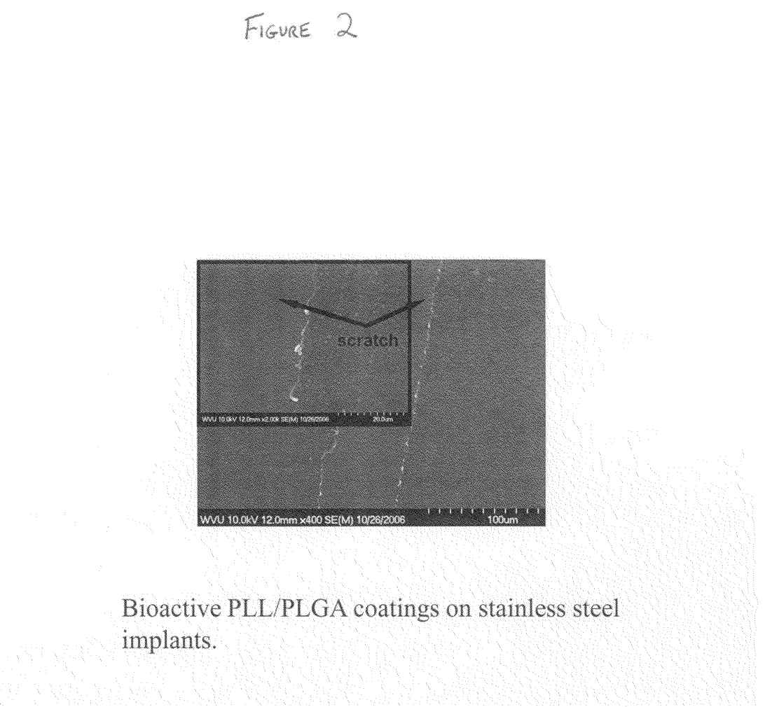

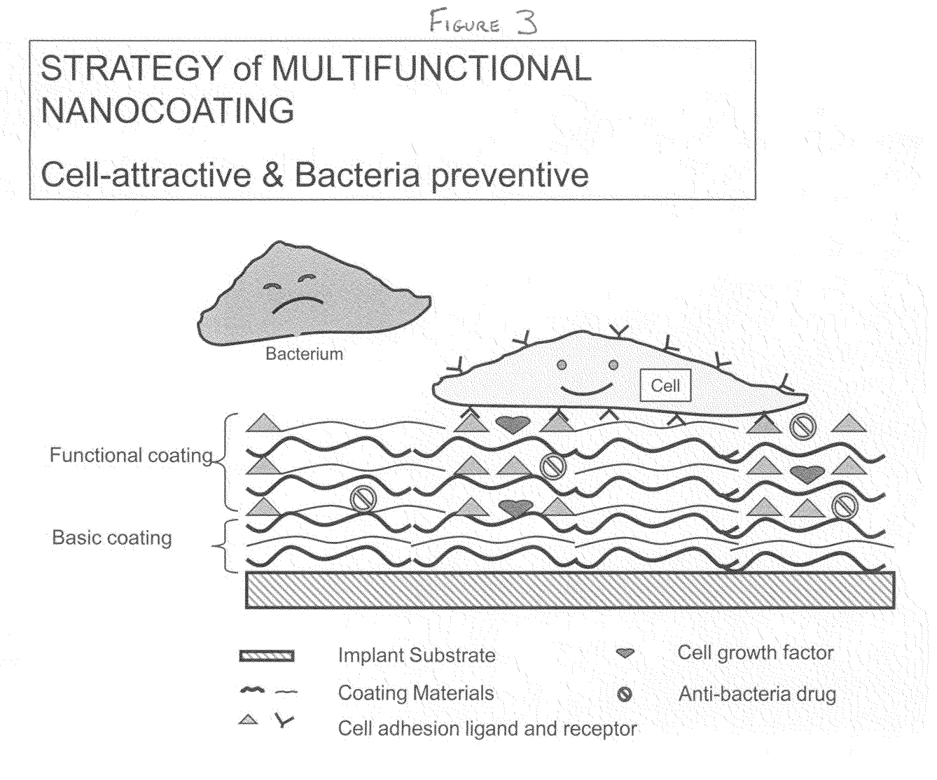

A first embodiment of the coating and method can be to use electrostatic self-assembly to create surface coating layers with beneficial characteristics for indwelling medical devices. Both organic and inorganic surfaces including metals, ceramics, and polymers amongst others may be coated with this method. One embodiment can be used to create biocompatible or bioactive (“biocompatible molecules” or “bioactive molecules” shall mean any molecule which provides biologically compatible, tolerant, or beneficial characteristics either independently or in combination with one or more other molecules or substances. For purposes of this application and any related divisionals or continuations, the terms “bioactive molecule” and “biocompatible molecule” shall both have the above definition. Furthermore, use of the singular or plural form of “bioactive molecule” shall be deemed to include both the singular and plural “bioactive molecule” and “bioactive molecules”) coatings which may be multilayered FIGS. 1 and 2. In another embodiment, bioactive molecules may be incorporated into the layer(s) of the coating and/or upon its outer surface. These bioactive molecules can be specifically tailored for the device, the device's placement, the patient, the environment, or any other reason to accomplish goals such as preventing infection, promoting healing, promoting growth, or even for benefits which may be unrelated to the implant's purpose, effectiveness, acceptance, or complications FIG. 3.

In an embodiment of this coating and method, electrostatic self-assembly may be used to incorporate bioactive molecules in a multi-layer structure such as ADAD . . . , where A is a positively or negatively charged layer created from one or more water soluble polyelectrolytes and D is a layer, oppositely charged to A, created from one or more water soluble polyelectrolytes. This electrostatic self-assembly is performed by any means known to one skilled in the art. The above mentioned polyelectrolytes may be any known to those skilled in the art, including but not limited to, polyethyleneimine (PEI), chitosan, poly(ally amine hydrochloride) (PAH), polylysine, poly(glutamic acid), albumin, interleukin-12, DNA, heparin, and alginate.

Bioactive molecules may be incorporated into or on the coating via several means. In one embodiment, a bioactive molecule, B, may be incorporated within layers A and/or D via some affinity for one or more of the polyelectrolytes within the particular layer. Such affinities may include, but are not limited to, hydrogen bonding, electrostatic forces, Van der Waals forces, hydrophobic forces, hydrophilic forces, and any other affinity known to those skilled in the art. A diagram of such a coating could be (A+B)(D+B)(A+B).

Another embodiment may incorporate bioactive molecules through use of a protein carrier molecule, C, which may be used to bind a bioactive molecule and form a coating layer. A carrier molecule may also have additional characteristics such as protecting the bioactive molecules, B, to which it is bound from denaturing, losing bioactivity, or otherwise being destroyed through incorporation in the coating, the method of coating, or its intended environments of storage or use. A diagram of such a coating could be A(B+C)A(B+C) where A is again a positively or negatively charged layer created from one or more water soluble polyelectrolytes. The bioactive molecule bound to a protein carrier complex, B+C, forms a layer next to A via certain affinity for A. Such affinities may include, but are not limited to, hydrogen bonding, electrostatic forces, Van der Waals forces, hydrophobic forces, hydrophilic forces, and any other affinity known to those skilled in the art. Another embodiment uses the carrier molecule to bind high molecular weight bioactive molecules into a still ultrathin coating using the same method as above.

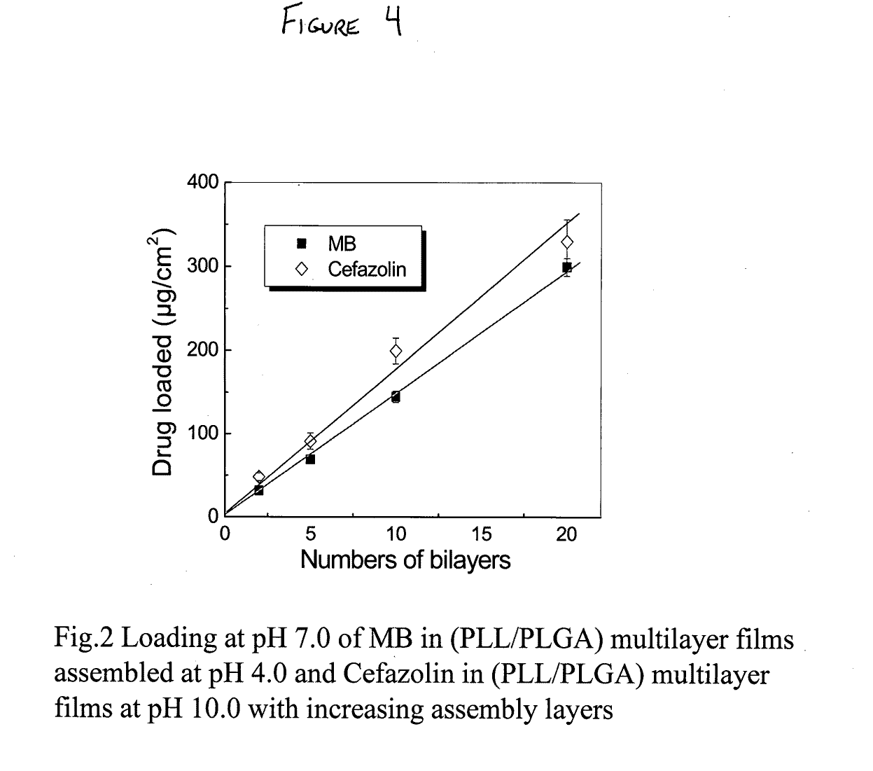

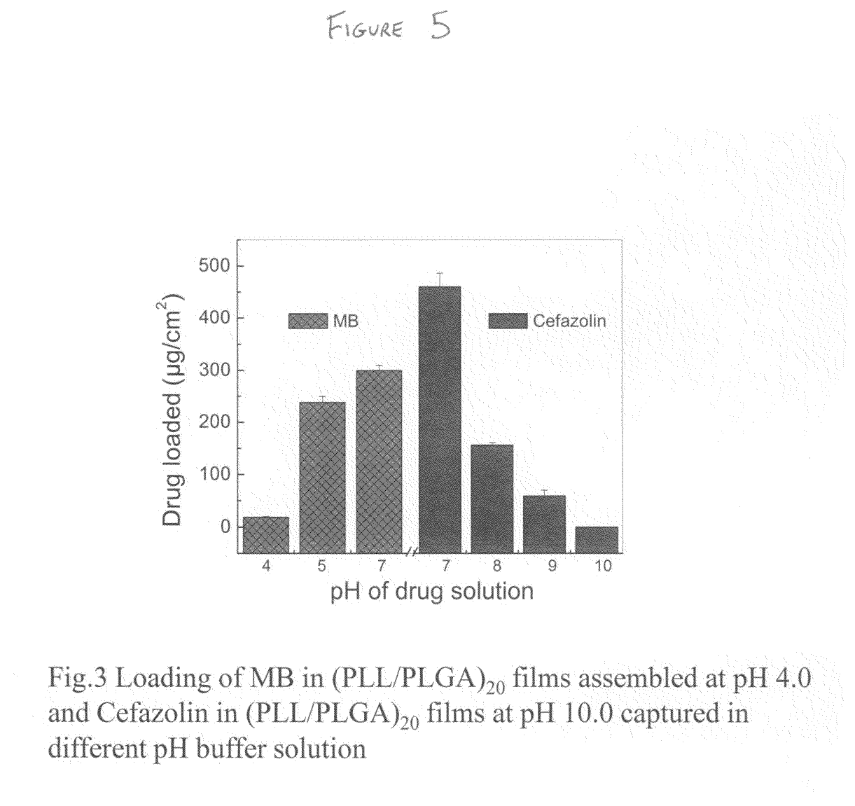

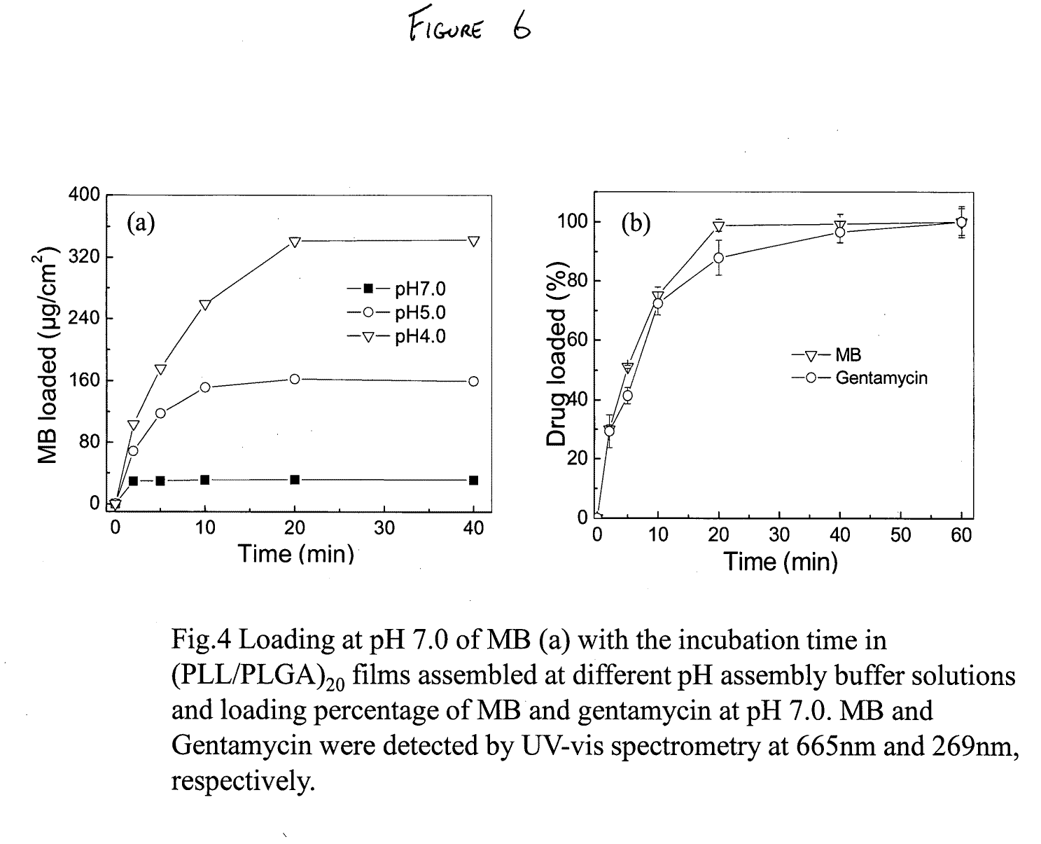

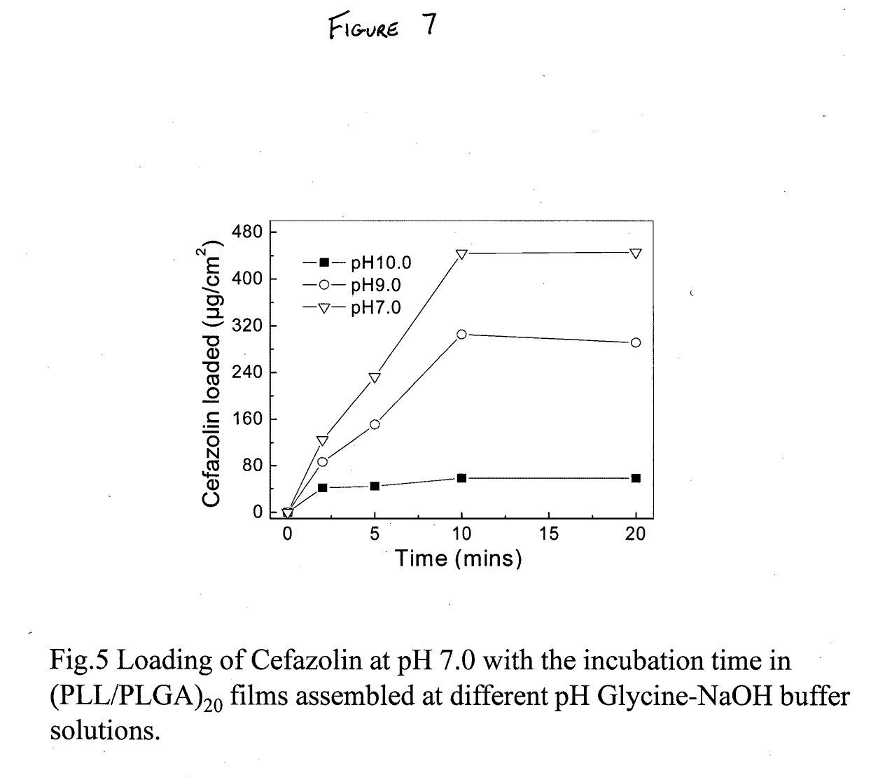

Yet another embodiment may incorporate bioactive molecules into an electrostatic self-assembly coating through post-treatment of the coating. Post-treatment of the coating includes submerging the coated surface for an effective time into an aqueous solution containing a bioactive molecule for which the coating has binding sites, activated through a raised or lowered pH level of the polyelectrolyte coating layers, for the bioactive molecule due to specific affinity with the bioactive molecule at certain pH FIGS. 4, 5, 6, and 7. Such raised or lowered pH levels may vary from a pH of about 2 to about 12. The bioactive molecules in the aqueous solution are drawn into the coating through diffusion and bound to the activated sites within the coating layers via some affinity. Such affinities may include, but are not limited to, hydrogen bonding, electrostatic forces, Van der Waals forces, hydrophobic forces, hydrophilic forces, and any other affinity known to those skilled in the art. Incorporation of bioactive molecules in this way may also provide for a sustained release of the bioactive molecules when such a coating is placed in a biological specimen of a pH differing from the coating and thus deactivating the bioactive molecule binding sites.

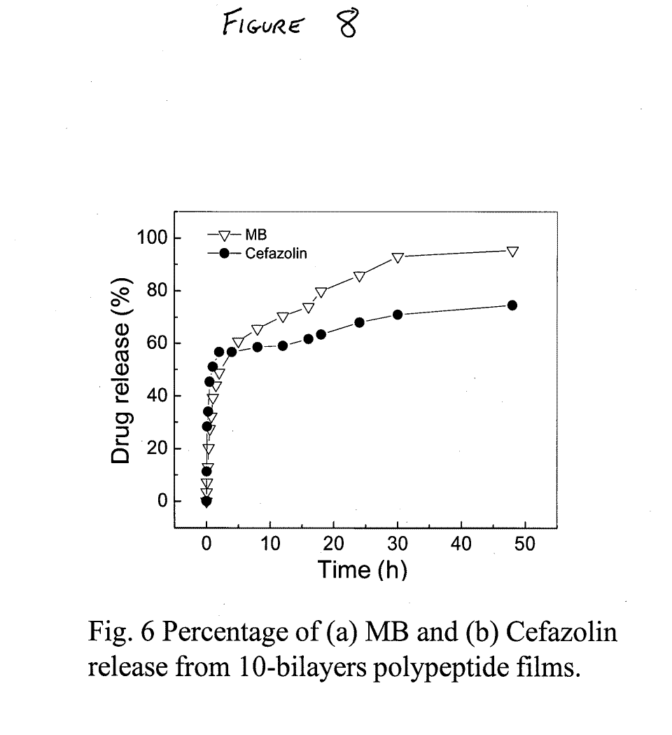

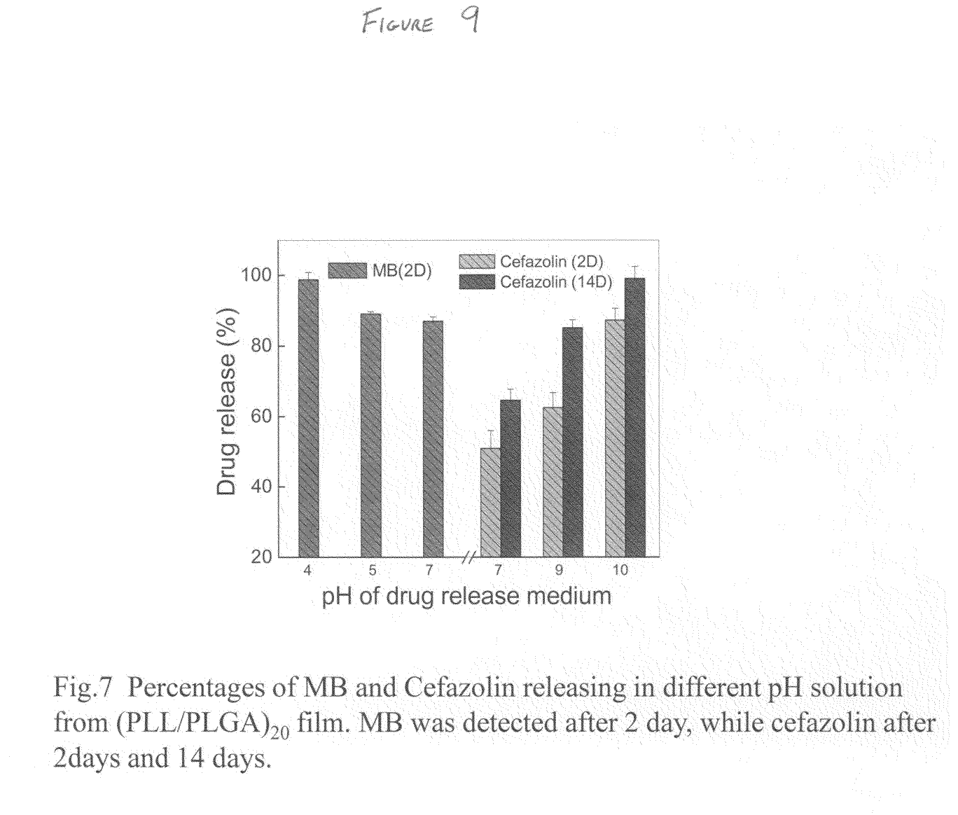

In an embodiment incorporating biocompatible or bioactive molecules, said bioactive molecules may be released from the coating by diffusion, swelling, or degradation of the coating. Release of bioactive molecules may be sustained over periods ranging from about a few hours to about one year or more. By varying the incorporation of bioactive molecules within different layers, the order of release of bioactive molecules may also be controlled FIGS. 8 and 9.

A further embodiment of the coating may contain Interleukin-12 to stimulate or induce type 1 helper T (Th1) cell responses and thereby stimulate cell-mediated immunity for infection prevention.

In another embodiment the structure of multilayer nanocoatings and the incorporation and release of bioactive molecules may be engineered through varying the thickness of coating layers from about 1 nm to about 500 nm. For example, in the case of an Interleukin-12 layer, each layer may be about 5 nm. The absorbance of the coating may be tuned with the coating structure, and the pre-layers of (PLL/PLGA)3 may increase absorbance of the (PLL/BSA)5 coating and also the release of Interleukin-12. The incorporation of Interleukin-12 may also be tuned by the concentration of Interleukin-12 in the BSA solution. More Interleukin-12 will be released from a layer prepared with a greater Interleukin-12 concentration. Incorporation of bioactive molecules can also be enhanced by simply increasing the number of coating layers in which said bioactive molecules are incorporated.

The process to fabricate the coatings comprises the steps of
5. Submerging the substrate material into a positively charged material (a) dissolved in an aqueous solution at a certain temperature, and incubating.
6. Rinsing the substrate material with the same aqueous buffer solution in first coating step or water to remove weakly bound material (a) on substrate surface and then drying.
7. Submerging the substrate material coated with material (a) into a negatively charged material (d) dissolved in another aqueous solution at a certain temperature, and incubating.
8. Rinsing the substrate material with the same aqueous buffer solution in the second coating step or water to remove weakly bound material (d) on substrate surface and then drying.

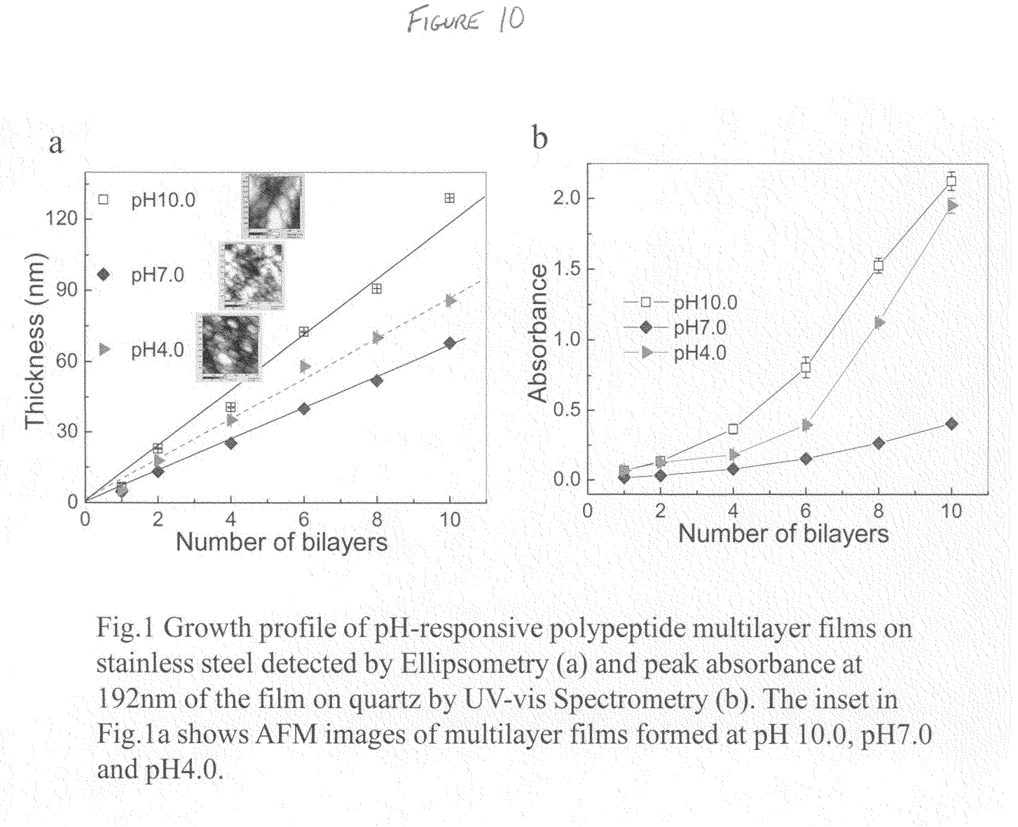

The entire above steps can be repeated to obtain multilayer coatings on substrate material FIG. 10. To incorporate bioactive molecules within the layers, the substrate material is coated with multilayers of material (a) and material (d) in bioactive molecules (b) containing solutions and then dried. Additionally, the bioactive molecules (b) can be incorporated into the steps above by placing the bioactive molecules within the positively and or negatively charged solutions in which the substrate is submerged The bioactive molecules (b) may also be bound to carrier proteins (c) which may provide beneficial binding or protection characteristics.

The substrate materials or surfaces to be coated may be inorganic or organic materials. Inorganic materials which may be coated with this method include but are not limited to titanium, stainless steel, glass, silica, ceramics or metal alloys. Organic materials which may be coated with this method include but are not limited to plastics, polymers.

The positively charged material (a) may be selected from water-soluble polyelectrolytes that are used in medical application such as polyethyleneimine (PEI), chitosan, poly(ally amine hydrochloride) (PAH) and polylysine, but are not limited to these examples.

The negatively charged material (d) can be selected from water-soluble polyelectrolytes that are used in medical application such as poly(glutamic acid), albumin, interleukin-12, DNA, heparin, and alginate, but are not limited to these examples.

The aqueous solution for material (a) and material (d) dissolving, coating temperature and incubating time in first and third coating steps may be varied to control the coating amount and morphology of material (a) and material (d) on the substrate surface.

The rinse solutions for material (a) dissolving, rinse time and drying times in the second and fourth coating steps are for remove of loosely-bound material (a) and (d) and aqueous solvent on the substrate surface, and may be varied significantly.

Bioactive molecules (b) may include but are not limited to water-soluble materials with biological function such as antimicrobial agents, drugs, antibiotics, cytokines, growth factors, growth inhibitors, silver, zinc oxide, anti-coagulants, Arg-Gly-Asp (RGD)-containing compounds, chemotherapeutic agents, quantum dots, genetic material, cells, vitamins, minerals and binding ligands.

The method may also be reversed such that a negatively charged material is used in the first and second steps, and a positively charged material is used in the third and fourth steps. Additionally, affinity forces other than electrostatic forces may be used in adjoining layers which may include, but are not limited to, hydrogen bonding, electrostatic forces, Van der Waals forces, hydrophobic forces, hydrophilic forces, and any other affinity known to those skilled in the art.

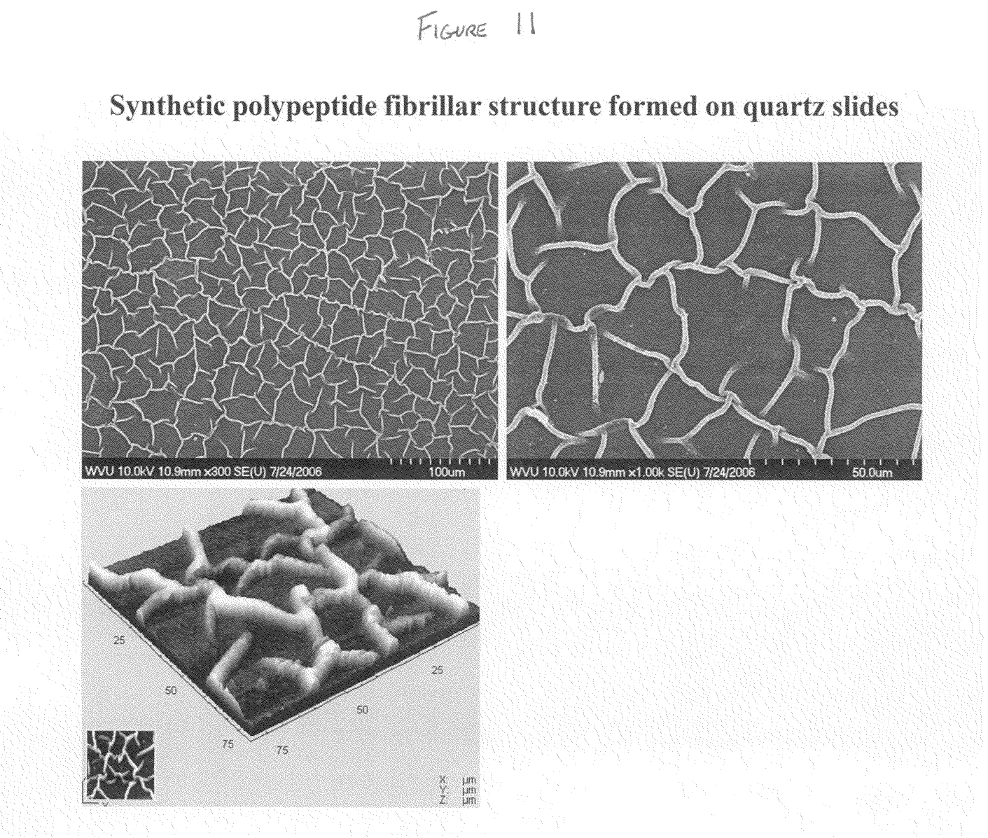

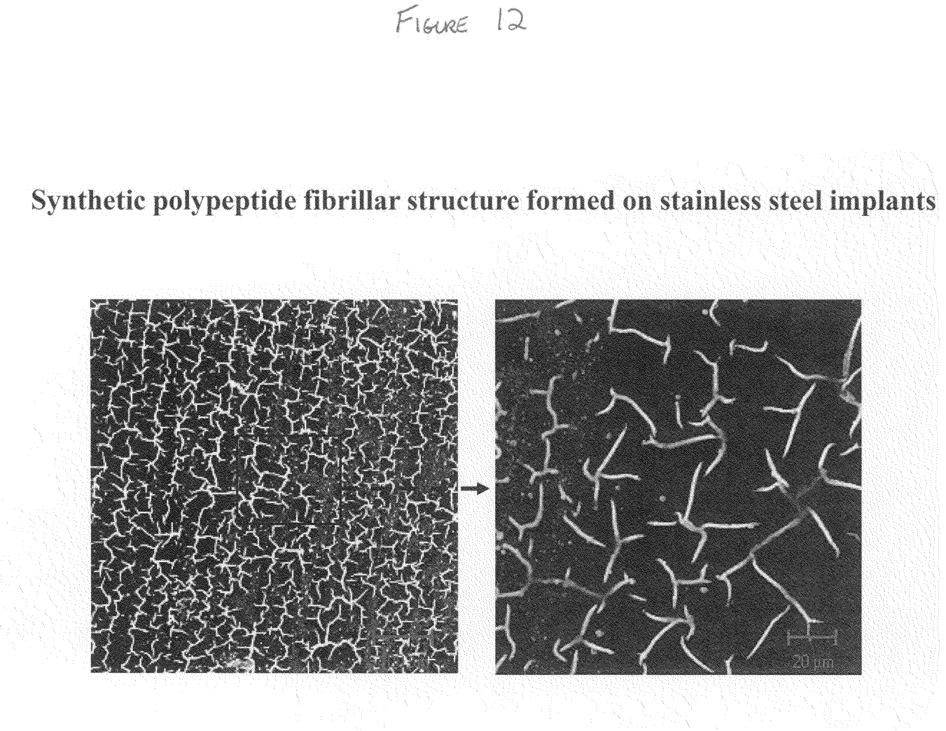

Yet another embodiment may have an extracellular matrix (ECM)-like surface FIGS. 11 and 12. ECM-like shall be defined as a having a structure similar to the structure of the extracellular matrix and/or fibronectin, an important member of extracellular matrix, but formed from synthetic materials (no fibronectin). The coating is made of non-ECM synthetic polymers and shows an ECM-like surface, permitting and encouraging implant-tissue interaction, bone attachment and promoting wound healing. High or low molecular weight bioactive molecules can also be incorporated into this ECM-like surface.

More specific embodiments are as follows:

### Example 1

### Interleukin (IL)-12 Loaded Infection Prevention Coatings

Commercial fracture fixation devices, Kirschner wires (K-wires), obtained from Smith & Nephew Inc. were used as a substrate material for coatings after rinsing in 70% alcohol. Poly(L-lysine) i.e. PLL and poly(L-glutamic acid) i.e. PLGA are dissolved in a phosphate buffer solution (PBS) with pH at 4.0, 5.0, 7.0, 9.0 or 10.0; interleukin-12 (IL-12) is dissolved in a 0.2% bovine serum albumin (BSA)-containing PBS of pH7.0; PBS is used as rinse solution.

Introducing K-wires into PLL solution for 20 minutes (mins) at 4° C., 10° C. or ambient temperature;

Rinsing K-wires with PBS, and then drying by N₂ gas gun;

Introducing PLL-coated K-wires in IL-12 solution for 20 mins,

Rinsing K-wires with PBS, and then drying by N₂ gas gun;

Introducing coated K-wires in PLL solution for 20 mins,

Rinsing K-wires with PBS, and then drying by N₂ gas gun;

Introducing coated K-wires in PLGA solution for 20 mins,

Rinsing K-wires with PBS, and then drying by N₂ gas gun;

Repeat the above steps for 5, 10, or 20 times, then dry by N₂ gas gun;

The substrate and solutions are sterilized by means known to one skilled in the art. All procedures were performed under sterile laminar airflow conditions (in a sterilized, closed glove box).

### Example 2

### ECM-Like Multilayer Coating on Substrates

Stainless steel, titanium disk and quartz slides were used as a substrate material for coatings.

PLL and PLGA are dissolved in glycine buffer solution with pH at 4.0, 5.0, 7.0, 9.0 or 10.0; glycine buffer solution is used as rinse solution.

Introducing substrate into PLL solution for 20 minutes at ambient temperature;

Rinsing substrate with glycine buffer solution, and then drying by N₂ gas gun;

Introducing PLL-coated substrates in PLGA solution for 20 mins,

Rinsing substrates with glycine buffer solution, and then drying by N₂ gas gun;

Repeat the above steps for 20, 40 or 80 times, then dry by N₂ gas gun;

### Example 3

### Cefazolin Incorporated Infection Preventive Coatings

PLL/PLGA-cefazolin multilayer coating on substrates

Stainless steel, titanium plates or K-wires, and quartz slides were used as a substrate material for coatings.

PLL and PLGA are dissolved in Tris-buffer solution with pH at 4.0, 5.0, 7.0, 9.0 or 10.0;

Tris-buffer solution is used as the rinse solution.

Introducing substrate into PLL solution for 20 minutes at ambient temperature;

Rinsing substrate with Tris-buffer solution, and then drying by N₂ gas gun;

Introducing PLL-coated substrate in PLGA solution for 20 mins,

Rinsing them with Tris-buffer solution, and then drying by N₂ gas gun;

Repeat the above steps for 1, 2, 5, 10, or 20 times, then drying by N₂ gas gun

Introducing the substrate coated with multilayers of PLL and PLGA in cefazolin containing Tris-buffer solutions for 1, 3, 8, 15, 30, 45, 60 or 90 min and then drying by N₂ gas gun.

Sterilize the coated substrate by means known to one skilled in the art. Coated substrate may then be placed in a medium containing Staphylococcus aureus (S. aureus) to check antibacterial properties.

### Example 4

### Gentamycin Incorporated Infection Preventive Coatings

PLL/PLGA-gentamycin multilayer coating on substrates

Stainless steel, titanium disk and quartz slides were used as a substrate material for coatings.

PLL and PLGA are dissolved in Tris-buffer solution with pH at 4.0, 5.0, 7.0, 9.0 or 10.0;

Tris-buffer solution is used as rinse solution.

Introducing substrate into PLL solution for 20 minutes at ambient temperature;

Rinsing substrate with Tris-buffer solution, and then drying by N₂ gas gun;

Introducing PLL-coated substrate in PLGA solution for 20 mins,

Rinsing substrate with Tris-buffer solution, and then drying by N₂ gas gun;

Repeat the above steps for 1, 2, 5, 10, or 20 times, then drying by N₂ gas gun;

Placing the coated substrate in gentamycin-containing Tris-buffer solution, then drying by N₂ gas gun.

Sterilize the coated substrate by means known to one skilled in the art. Coated substrate may then be placed in a medium containing S. aureus to check antibacterial properties.

### Example 5

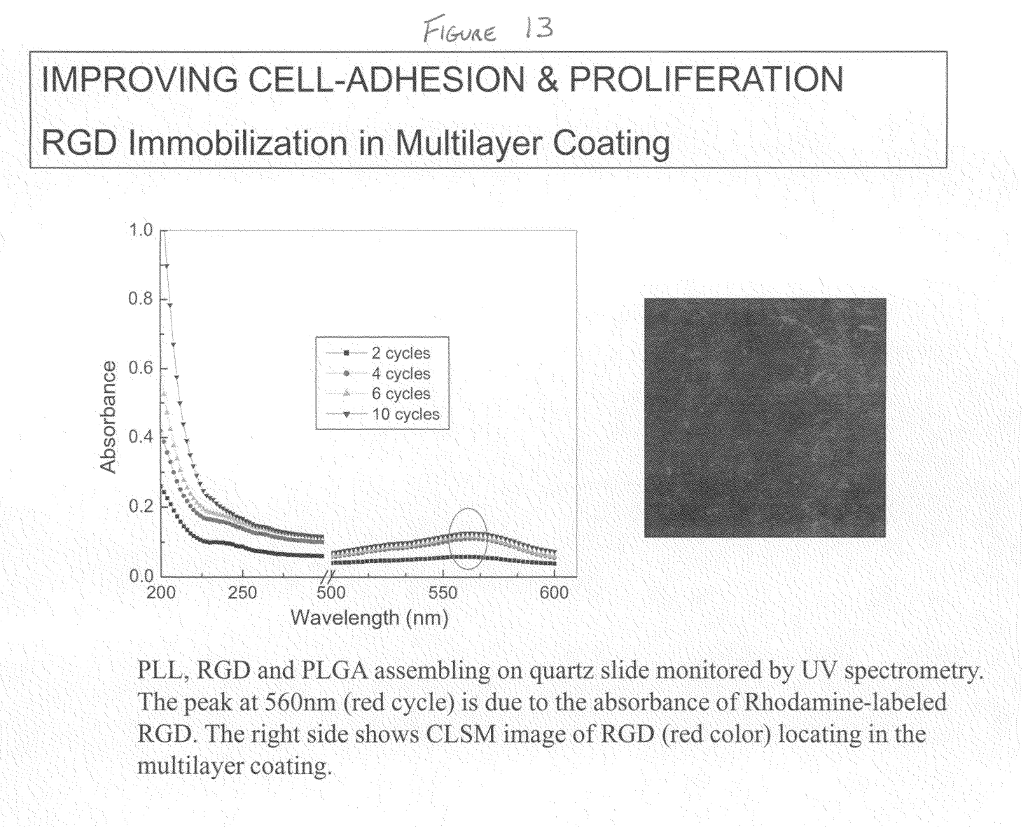

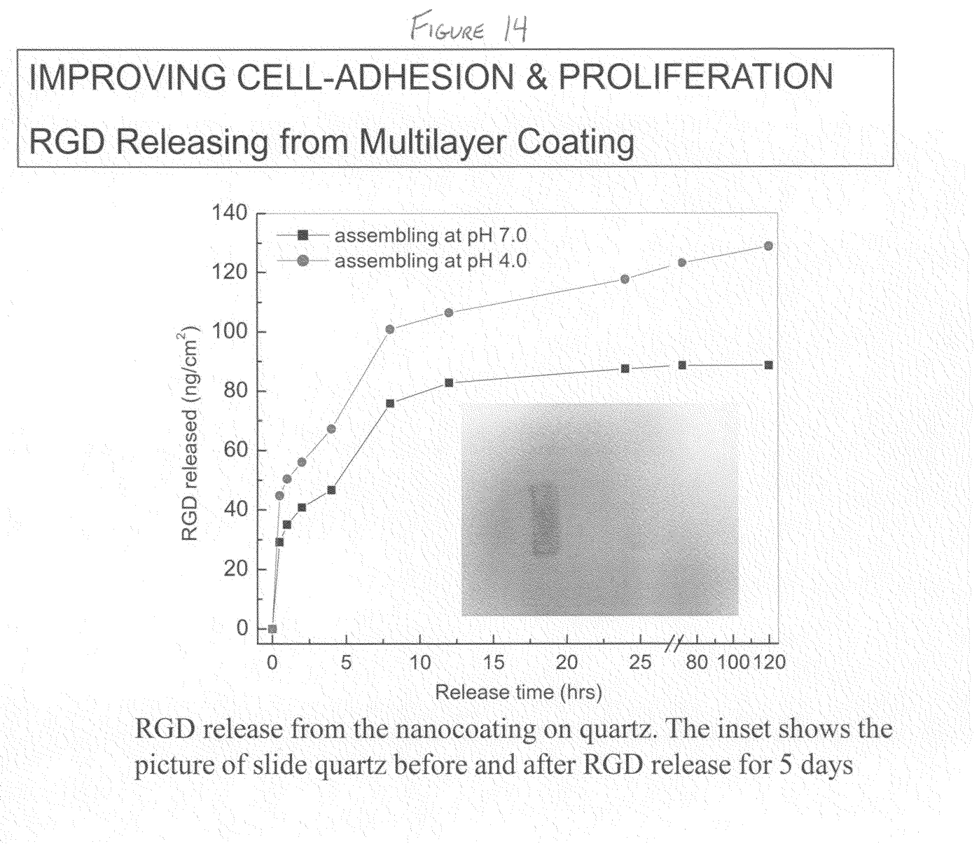

### RGD Incorporated Bone Cell Adhesion Promoting Coatings FIGS. 13 and 14

PLL/PLGA-RGD multilayer coating on substrates

Stainless steel, titanium disk, silicon and quartz slides were used as a substrate material for coatings.

PLL, RGD and PLGA are dissolved in Tris-sodium acetate buffer solution with pH at 4.0, 5.0, or 7.0; Tris-sodium acetate buffer solution is used as rinse solution.

Introducing substrate into PLL solution for 20 minutes at ambient temperature;

Rinsing substrate with the Tris-sodium acetate buffer solution, and then drying by N₂ gas gun;

Introducing PLL-coated substrates in RGD solution for 20 mins,

Rinsing substrate with the Tris-sodium acetate buffer solution, and then drying by N₂ gas gun;

Introducing coated substrate into PLL solution for 20 minutes;

Rinsing substrate with Tris-sodium acetate buffer solution, and then drying by N₂ gas gun;

Introducing coated substrates in PLGA solution for 10 mins,

Rinsing substrate with Tris-sodium acetate buffer solution, and then drying by N₂ gas gun;

Repeat the above steps for 1, 2, 5, 10, or 20 times, then drying by N₂ gas gun;

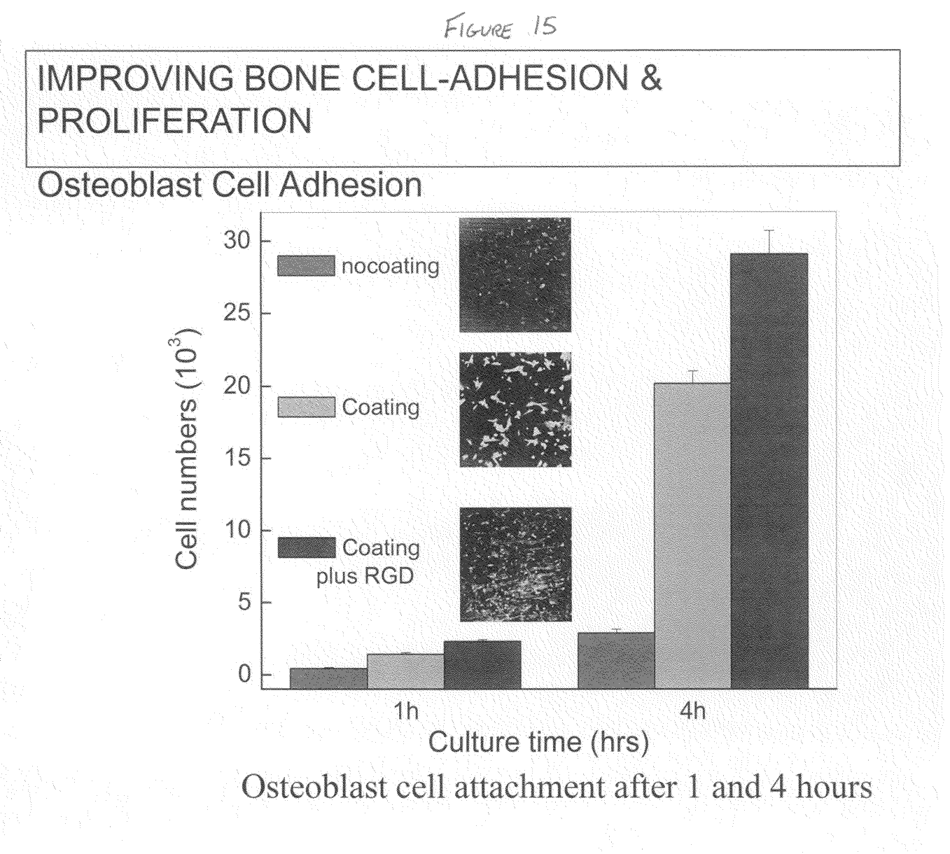

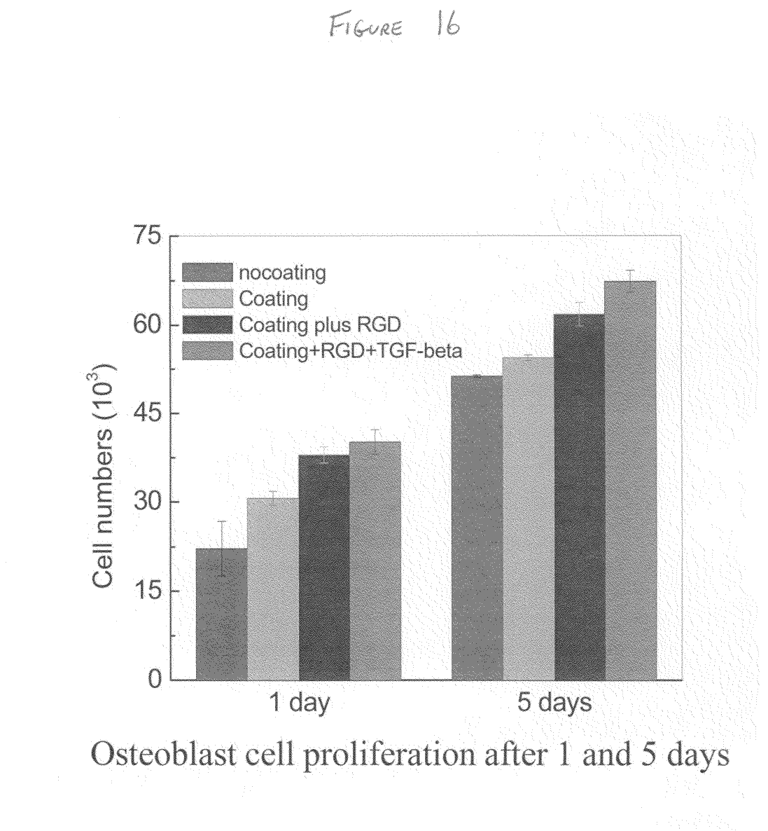

Sterilize the coated substrate by means known to one skilled in the art. Coated substrate may then be placed in a medium containing osteoblast cells to check cell adhesion and proliferation FIGS. 15 and 16.

### Example 6

### RGD and Transforming Growth Factor (TGF)-β1 Incorporated Bone Cell Adhesion and Growth Promoting Coatings

PLL/PLGA-RGD-TGF-β1 multilayer coating on substrates

Stainless steel, titanium disk, silicon and quartz slides were used as a substrate material for coatings.

PLL, RGD and PLGA are dissolved in Tris-sodium acetate buffer solution with pH at 4.0, 5.0, or 7.0; TGF-131 is dissolved in a 0.2% BSA-containing PBS of pH7.0; Tris-sodium acetate buffer solution is used as rinse solution.

Introducing substrate into PLL solution for 20 minutes at ambient temperature;

Rinsing substrate with Tris-sodium acetate buffer solution, and then drying by N₂ gas gun;

Introducing PLL-coated substrates in RGD solution for 20 mins,

Rinsing substrate with Tris-sodium acetate buffer solution, and then drying by N₂ gas gun;

Introducing coated substrate into PLL solution for 20 minutes;

Rinsing substrate with Tris-sodium acetate buffer solution, and then drying by N₂ gas gun;

Introducing coated substrates in PLGA solution for 10 mins,

Rinsing substrate with Tris-sodium acetate buffer solution, and then drying by N₂ gas gun;

Repeat the above steps for 1, 2, 5, 10, or 20 times, then drying by N₂ gas gun;

Placing the coated substrate in TGF-131-containing PBS with 1 mg/ml BSA as a protein carrier, then drying by N₂ gas gun.

Sterilize the coated substrate by means known to one skilled in the art. Coated substrate may then be placed in a medium containing osteoblast cells to check cell adhesion and proliferation.

### Example 7

### Gentamycin and RGD Incorporated Bone Cell Adhesion Promoting and Infection Preventive Coatings

PLL/PLGA-gentamycin-RGD multilayer coating on substrates

Stainless steel, titanium disk, and quartz slides were used as a substrate material for coatings.

PLL, RGD and PLGA are dissolved in Tris-buffer solution with pH at 4.0, 5.0, or 7.0;

Tris-buffer solution is used as rinse solution.

Introducing substrate into PLL solution for 20 minutes at ambient temperature;

Rinsing substrate with Tris-buffer solution, and then drying by N₂ gas gun;

Introducing PLL-coated substrates in RGD solution for 20 mins,

Rinsing substrate with Tris-buffer solution, and then drying by N₂ gas gun;

Introducing coated substrate into PLL solution for 20 minutes;

Rinsing substrate with Tris-buffer solution, and then drying by N₂ gas gun

Introducing coated substrates in PLGA solution for 20 mins,

Rinsing substrate with Tris-buffer solution, and then drying by N₂ gas gun;

Repeat the above steps for 1, 2, 5, 10, 20 or 40 times, then drying by N₂ gas gun;

Placing the coated substrate in gentamycin-containing Tris-buffer solution, and then drying by N₂ gas gun.

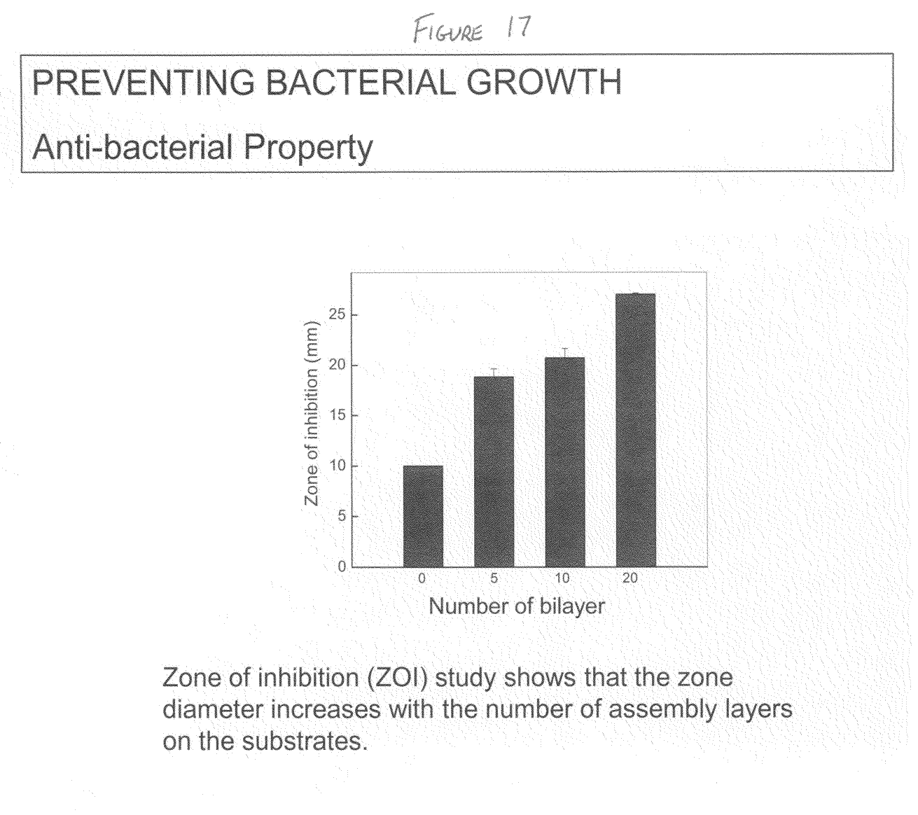

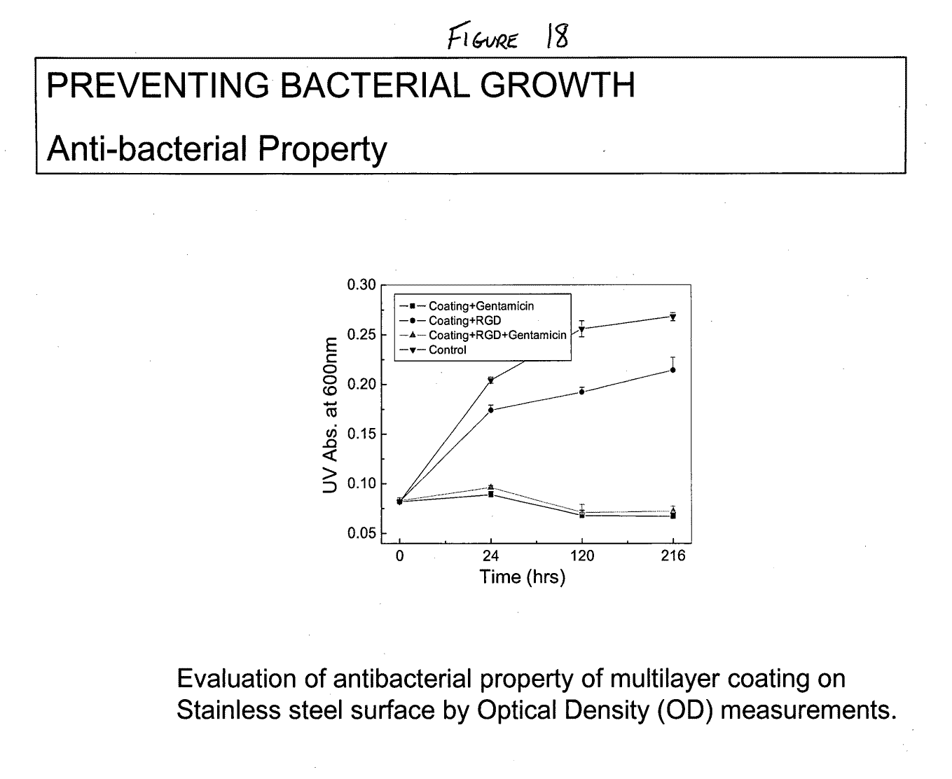

Sterilize the coated substrate by means known to one skilled in the art. Coated substrate may then be placed in a medium containing osteoblast cells to check cell adhesion and proliferation and/or in a medium containing S. aureus to check antibacterial properties FIGS. 17 and 18.

### Example 8

### Antibiotic, RGD and Growth Factor Incorporated Bone Cell Adhesion & Growth Promoting and Infection Preventive Coatings

PLL/PLGA-gentamycin-RGD-TGF-β1 multilayer coating on substrates

Stainless steel, silicon and quartz slides were used as a substrate material for coatings.

PLL, RGD and PLGA are dissolved in Tris-sodium acetate buffer solution with pH at 4.0, 5.0, or 7.0; TGF-β1 is dissolved in a 0.2% BSA-containing PBS of pH7.0; Tris-sodium acetate buffer solution is used as rinse solution.

Introducing substrate into PLL solution for 20 minutes at ambient temperature;

Rinsing substrate with Tris-sodium acetate buffer solution, and then drying by N₂ gas gun;

Introducing PLL-coated substrates in RGD solution for 20 mins,

Rinsing substrate with Tris-sodium acetate buffer solution, and then drying by N₂ gas gun;

Introducing coated substrate into PLL solution for 20 minutes;

Rinsing substrate with Tris-sodium acetate buffer solution, and then drying by N₂ gas gun;

Introducing coated substrates in PLGA solution for 10 mins,

Rinsing substrate with Tris-sodium acetate buffer solution, and then drying by N₂ gas gun;

Repeat the above steps for 1, 2, 5, 10, or 20 times, then drying by N₂ gas gun;

Placing the coated substrate in gentamycin-containing PBS, then drying by N₂ gas gun;

Placing the coated substrate in TGF-β1-containing PBS with 1 mg/ml BSA as a protein carrier, then drying by N₂ gas gun.

Sterilize the coated substrate by means known to one skilled in the art. Coated substrate may then be placed in a medium containing osteoblast cells to check cell adhesion and proliferation and/or in a medium containing S. aureus to check antibacterial properties.

### Example 9

### Cell Adhesion Promoting and Infection Preventive Coatings with ECM-Like Structure

ECM-like plus cefazolin multilayer coating on substrates

Stainless steel, titanium disk and quartz slides were used as a substrate material for coatings.

PLL and PLGA are dissolved in glycine buffer solution with pH at 7.0, 9.0 or 10.0; glycine buffer solution is used as rinse solution.

Introducing substrate into PLL solution for 20 minutes at ambient temperature;

Rinsing substrate with glycine buffer solution, and then drying by N₂ gas gun;

Introducing PLL-coated substrates in PLGA solution for 20 mins,

Rinsing substrate with glycine buffer solution, and then drying by N₂ gas gun;

Repeat the above steps for 20 or 40 times, then drying by N₂ gas gun;

Placing the coated substrate in cefazolin-containing PBS of pH 7.0, then drying by N₂ gas gun.

Sterilize the coated substrate by means known to one skilled in the art. Coated substrate may then be placed in a medium containing osteoblast cells to check cell adhesion and/or in a medium containing S. aureus bacteria to check antibacterial properties.

What is claimed is:

1. A coating for a surface comprising:
an electrostatic self-assembly creating one or more coating layer(s) (i) wherein one or more high molecular weight bioactive molecule(s) is bound to a protein carrier wherein said protein carrier or said bound bioactive molecule is further bound to one or more of said coating layer(s) and/or (ii) wherein one or more low molecular weight bioactive molecule(s) is/are incorporated within one or more of said layers or later introduced to said layer or layers by post-treatment.

2. The coating of claim 1 wherein said coating is for the surface of an indwelling medical device.

3. The coating of claim 1 wherein said coating is for a metal, ceramic, or polymer surface.

4. The coating of claim 1 wherein one or more of said high molecular weight bioactive molecule(s) is/are chosen from the group consisting of Interleukin-12, drugs, antibiotics, cytokines, growth factors, growth inhibitors, genetic material, cells, and binding ligands.

5. The coating of claim 1 wherein said protein carrier(s) is/are chosen from the group consisting of bovine serum albumin, sterol carrier proteins, glycolipid transfer proteins, transmembrane proteins, and acyl carrier proteins.

6. The coating of claim 1 wherein said protein carrier(s) also protects the bioactivity of said high molecular weight bioactive molecule(s) and/or protects said high molecular weight bioactive molecule(s) from denature and/or destruction.

7. The coating of claim 1 wherein one or more of said low molecular weight bioactive molecule(s) is/are chosen from the group consisting of drugs, antibiotics, cytokines, growth factors, growth inhibitors, silver, zinc oxide, antimicrobial agents, anti-coagulants, RGD-containing compounds, chemotherapeutic agents, quantum dots and binding ligands.

8. The coating of claim 1 wherein one or more of said high-molecular weight bioactive molecule(s) or low-molecular weight bioactive molecule(s) are bound to the said layer(s) through covalent bonds.

9. The coating of claim 1 wherein the sustained release of one or more of said high-molecular weight bioactive molecule(s) or low-molecular weight bioactive molecule(s) is controlled by diffusion, and environmental pH.

10. The coating of claim 1 wherein one or more of said coating layer(s) are biodegradable.

11. The coating of claim 10 wherein the rate of decay of said biodegradable layer(s) may range from about several hours to about more than one year.

12. The coating of claim 10 wherein the sustained release of one or more of said high-molecular weight bioactive molecule(s) or low-molecular weight bioactive molecule(s) is controlled by one or more of the rate of diffusion, environmental pH, and the rate of decay of said biodegradable layer(s).

13. The coating of claim 10 wherein the sequence of sustained release of two or more of said high-molecular weight bioactive molecule(s) or low-molecular weight bioactive molecule(s) is controlled by one or more of the size of the bioactive molecule(s), placement within sequential said biodegradable coating layer(s), and the interaction between the bioactive molecule(s) and the coating layer(s).

14. The coating of claim 4 wherein said high molecular weight bioactive molecules are Interleukin-12 and one or more drug molecules that can enhance wound healing wherein said high molecular weight bioactive molecules stimulate a type 1 helper T (Th1) cell response.

15. The coating of claim 1 further comprising an extracellular matrix (ECM)-like outer surface with a structure similar to the fibrillar structure of fibronectin, an extracellular matrix component, but not containing fibronectin or any extracellular matrix components.

16. The coating of claim 15 wherein said ECM-like structure is made of poly(lysine) and poly(glutamic acid).

17. A method of coating a surface comprising:
a. introducing said surface into a solution of one or more positively charged water-soluble polyelectrolytes at a pH ranging from about 2 to about 12 and at a temperature ranging from about 4° C. to about 80° C. and incubating said surface for about 0.5 to about 60 minutes;
b. Rinsing said surface with said buffer solution or water to remove weakly bound material on said surface and dry by means known to one skilled in the art;
c. Introducing said surface into a solution of one or more negatively charged water-soluble polyelectrolytes at a pH ranging from about 2 to about 12 and at a temperature ranging from about 4° C. to about 80° C. and incubating said surface for about 0.5 to about 60 minutes; and
d. Rinsing said surface with said buffer solution or water to remove weakly bound material on said surface and passively or actively drying said surface.

18. The method of claim 17 wherein one or more of said positively charged water-soluble polyelectrolyte(s) is/are chosen from the group consisting of proteins, polypeptides, polylysine, chitosan, polyethyleneimine, polyacrylamide, poly(ally amine hydrochloride), and poly(diallyldimethylammonium chloride).

19. The method of claim 17 wherein one or more of said negatively charged water-soluble polyelectrolyte(s) is/are chosen from the group consisting of proteins, polypeptides, DNA, poly(glutamic acid), albumin, interleukin-12, heparin, gelatin, alginate, dextran sulfate, hyaluronan, chondroitin, poly(acrylic acid), poly(styrenesulfonate), poly(vinylsulfonate), and nanoparticles.

20. The method of claim 17 wherein said method is repeated any number of times to obtain multilayer coatings on said surface.

21. The method of claim 17 wherein one or more bioactive molecule(s) is/are impregnated into the said surface coating by being introduced to said positively charged aqueous solution and/or said negatively charged aqueous solution.

22. The method of claim 17 wherein one or more bioactive molecule(s) is/are impregnated into said surface coating by placing said surface in solutions of said bioactive molecule(s) for about 5 seconds to about 1 week and drying.

23. The method of claim 17 wherein on or more bioactive molecule(s) is/are bound to a protein carrier wherein said protein carrier is bound to one or more of said coating layers.
